(19) United States
(12) Patent Application Publication
(10) Pub. No.: US 2024/0065896 A1
(43) Pub. Date: Feb. 29, 2024

(54) Non-Invasive Infused Activated Charcoal Medical Patch

(71) Applicant: Wanda Manns-Griffin, Randallstown, MD (US)

(72) Inventor: Wanda Manns-Griffin, Randallstown, MD (US)

(21) Appl. No.: 17/897,300

(22) Filed: Aug. 29, 2022

(51) Int. Cl. A61F 13/00; A61F 13/02

(52) U.S. Cl. CPC A61F 13/00063; A61F 13/0246; A61F 2013/00906; A61F 2013/0091

(57) ABSTRACT

The present invention relates to an improved non-invasive transdermal medical patch. The medical patch is designed to attach to an ailing area of a body of a user for relieving chronic aches and pains. The medical patch comes in a variety of shapes and sizes, and is a non-invasive medical patch. The medical patch features a central bandage area infused with active charcoal, along with a recessed area that can receive ointments such as opioid medications. The patch uses a combination of the natural ingredient of activated charcoal and the medicated ointment for providing quick and easy relief from pain. The activated charcoal can be dried or lyophilized and can be moistened with water and then applied to an ailing body area of the user.

### FIELD OF THE INVENTION

The present invention relates generally to the field of transdermal delivery patches. Transdermal is a route of administration wherein active ingredients are delivered across the skin for systemic distribution. Examples include transdermal patches used for medicine delivery. The drug can be administered in the form of a patch or ointment that delivers the drug into the circulation for systemic effect. More specifically, the present invention relates to a medical patch for relieving chronic aches and pains. The medical patch comes in a variety of shapes and sizes and is a non-invasive medical patch that can be attached to any ailing area of the body. The medical patch features a central bandage area infused with active charcoal along with a recessed area that can receive ointments such as opioid medications. The activated charcoal can be dried or lyophilized and can be moistened with water and then applied to an ailing body area of the user. The patch can be specifically designed for the hands and toes and is especially effective for pain relief due to arthritis. Accordingly, the present disclosure makes specific reference thereto. Nonetheless, it is to be appreciated that aspects of the present invention are also equally applicable to other like applications, devices, and methods of manufacture.

### BACKGROUND

By way of background, individuals suffer pain throughout different areas of their body. Pain can be due to a variety of reasons such as wear and tear of tissues and muscles, physical injuries, infection, and/or the failure of the blood vessels surrounding the nerves. Pain is also caused due to the nerves that are part of the nervous system of the human body. Although some types of pain can be bearable and can be tolerated without taking any medication, some chronic and acute pain is unbearable and requires periodic medication. Some individuals prefer taking oral medications daily and multiple times in a day, but these medications are not safe for prolonged use and can cause side effects. Some individuals may end up taking injections when they are not able to take oral medications periodically.

Also, diseases of the joints pose a significant burden on the patient as mobility of the legs or fingers may be significantly affected. A common disease of the joints is arthritis, and more than 100 different forms of arthritis are known. The arthritis may result in trauma to the joints, infection of the joints, or the like. Patients of arthritis suffer from joint pain which is often long lasting and may be localized to the affected joint(s). Usually, treatment of arthritis includes physical therapy, lifestyle changes, orthopedic bracing, medications and occasionally joint replacement surgery. Individuals are reluctant to take oral medicines for such diseases as they end up taking them lifelong, which can cause other health issues over time.

The oral medications for treating and providing relief from pain are generally opioids, and their periodic use can cause side effects, such as constipation, nausea, sedation and more. Further, taking such oral medications can cause damage to the kidneys and the liver over long periods of time. The oral opioid medications can also weaken the immune system of the person.

Also, individuals take common pain killers without any prescription and consultation from doctors, and this causes side effects and can cause addiction to the person which can lead to serious health problems.

Nowadays, individuals also use medical patches for treating pain. A medical patch is a bandage-like transdermal patch that delivers pharmacological agents into the body through the skin. These patches deliver medications topically, where they are absorbed by the skin. However, such patches deliver only a single medication and cannot be used with more than one medication. Multiple ointments and medications cannot be provided by a single medical patch which reduces the effectiveness and utility of such a medical patch. Therefore, individuals desire an improved medical patch that overcomes the aforementioned problems.

Therefore, there exists a long felt need in the art for an improved medical patch that effectively provides relief from chronic and acute pains. There is also a long felt need in the art for an improved medical patch that eliminates the need to take periodic oral medication. Additionally, there is a long felt need in the art for an improved medical patch that can provide more than one ointment or medication. Moreover, there is a long felt need in the art for an improved medical patch that provides relief from pain of nerves, muscles, and tissues. Furthermore, there is a long felt need in the art for an improved medical patch that can be uniquely designed for application on different body parts, especially joints. Finally, there is a long felt need in the art for an improved medical patch that effectively provides quick and easy pain relief on any area of the body.

The subject matter disclosed and claimed herein, in one embodiment thereof, comprises a non-invasive medical patch for providing transdermal treatment for relieving pain to a target site of a body. The patch includes a central bandage area infused with active charcoal, a recessed area for receiving an ointment or additional medication wherein both the central bandage area and the recessed area are non-adhesive areas. The patch further includes adhesive areas adjacent on both sides of the central bandage area allowing the patch to stick to the skin of a target area of the body of a human and topically apply the activated charcoal and the ointment for quick and easy pain relief. The central bandage area can be moistened with water for activating the infused dried or lyophilized active charcoal before applying to the skin of the target area.

In this manner, the improved medical patch of the present invention accomplishes all of the forgoing objectives and provides a relatively safe, convenient and easy solution that offers a way to achieve relief from aches and pains on any area of the body. The patch can be applied to the target part of the body for a desired time period and can be applied with ointments for providing an effective and customized medication. Charcoal infused in the bandage can be used with the additionally applied ointment to topically apply the medication to the body.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a medical patch for providing medication for a pain treatment to a target site of a body in a transdermal manner. The patch further comprises a central bandage area infused with active charcoal, a recessed area disposed on the central bandage area for receiving an ointment or additional medication, wherein both the central bandage area and the recessed area are non-adhesive areas. The patch further includes adhesive areas adjacent on both sides of the central bandage area allowing the patch to stick to skin of a target area of the body of a human and topically apply the activated charcoal and the ointment for quick and easy pain relief. The central bandage area can be moistened with water for activating the infused charcoal before applying to the skin of the target area.

It is also disclosed that the patch, may include the dried or lyophilized activated charcoal composition for transdermal delivery of the activated charcoal in a known, accurate, and controlled dosage when it is moistened by water before application to the skin. According to the invention, the patch provides uniform and even distribution of the activated charcoal and the recessed medication, thereby highly improving transdermal delivery and bioavailability.

In yet another embodiment of the present invention, the recessed area for receiving the ointment or medication can also be disposed around the periphery of the central bandage area in addition to being disposed over the central bandage area.

In yet another embodiment, active charcoal infused in the patch can be mixed with additional ointments for application to the skin of the target area of the body.

In yet another embodiment, a method for providing relief from arthritis is described. The method includes providing a non-invasive and flexible medical patch to attach to a body part of a user, the patch including infused activated charcoal and a recessed area for receiving a pain relief medication, attaching the medical patch to the target body part and topically delivering the activated charcoal and the relief medication to the nerves, tissues, and muscles of the target body part for treating inflammation and pain, wherein the activated charcoal promotes circulation and draws out toxins as the toxins bind to the activated charcoal while the medication provides quick relief.

In yet another embodiment of the present invention, medication that is topically delivered can be one or more of buprenorphine, fentanyl, Duragesic, or any other opioid pain medication.

In yet another embodiment of the present invention, 5 to 10 micrograms of opioid medication can be infused per hour with use of patches as described supra.

In yet another embodiment of the present invention, a method of treating pain in a body part is disclosed. The method includes the steps of providing a medical patch that is configured to topically deliver a natural ingredient and a medication, applying the medical patch to an ailing target area of the body, and topically delivering the infused activated charcoal and an opioid medication to the ailing target area.

In yet another embodiment of the present invention, the patch can be used for one or more of the following of the alleviations of disease, for pain relief, for prophylactic care of the body and for improving the fitness of a human body.

The medical patch of the present invention is employed to provide relief naturally and medically from the pain, and enables active charcoal within the patch to mix with ointments for application to the skin. Further, the ointment can be infused or applied on the patch prior to application as desired. Additionally, the medical patch can start to relieve pain within 5 minutes of application of the patch on an ailing area of the body and can be easily applied for up to 48 hours without any skin irritation or rashes. The patch is waterproof and comes in both reusable and disposable variants.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of an improved medical patch of the present invention in accordance with the disclosed architecture;

FIG. 2A illustrates a rear perspective view of another potential embodiment of the transdermal medical patch device of the present invention in accordance with the disclosed architecture;

FIG. 2B illustrates a front perspective view of the another potential embodiment of the transdermal medical patch device of the present invention in accordance with the disclosed architecture;

FIG. 3A illustrates a rear perspective view of yet another potential embodiment of the transdermal medical patch device of the present invention in accordance with the disclosed architecture;

FIG. 3B illustrates a front perspective view of the yet another potential embodiment of the transdermal medical patch device of the present invention in accordance with the disclosed architecture; and

FIG. 4 illustrates a flow diagram showing exemplary steps performed for a method of providing relief from pain using one potential embodiment of the medical patch of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these q details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for an improved medical patch that effectively provides relief from chronic and acute pains. There is also a long felt need in the art for an improved medical patch that eliminates the need to take periodic oral medications. Additionally, there is a long felt need in the art for an improved medical patch that can provide more than one ointment or medication. Moreover, there is a long felt need in the art for an improved medical patch that provides relief from the pain of nerves, muscles and tissues. Furthermore, there is a long felt need in the art for an improved medical patch that can be uniquely designed for application on different body parts, especially joints. Finally, there is a long felt need in the art for an improved medical patch that effectively provides quick and easy pain relief on any area of the body.

The present invention, in one exemplary embodiment, is a method for providing relief from arthritis. The method includes providing a non-invasive and flexible improved medical patch to attach to an ailing body part of a user; the patch including infused, activated charcoal and a recessed area for receiving a pain relief medication. The method further includes attaching the medical patch to the target body part and topically delivering the activated charcoal and the relief medication to the nerves, tissues and muscles of the target body part for treating inflammation and pain, wherein the activated charcoal promotes circulation and draws out toxins as the toxins bind to the activated charcoal and the medication provides quick relief.

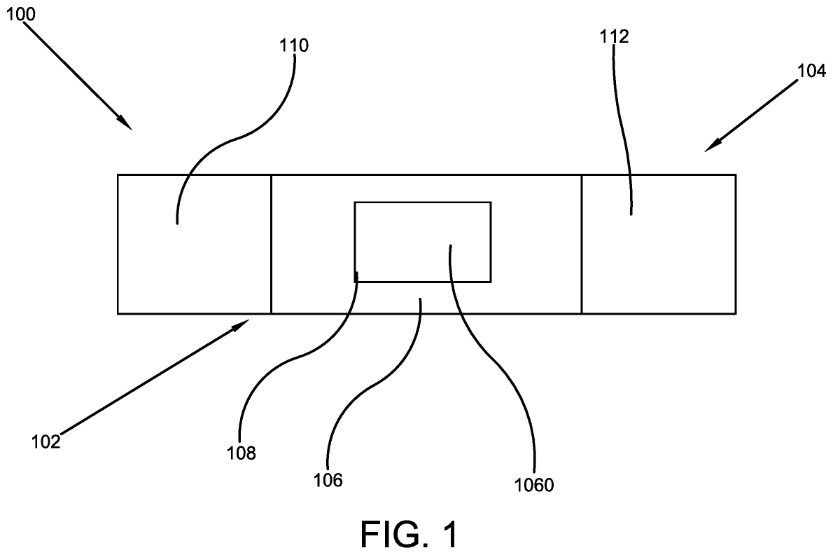

Referring initially to the drawings, FIG. 1 illustrates a perspective view of one potential embodiment of an improved medical patch of the present invention in accordance with the disclosed architecture. The medical patch 100 of the present embodiment is used for relieving chronic aches and pains. The patch 100 can be applied to skin of an ailing area of a body of a human for topically delivering the medication to the nerves, tissues and muscles. More specifically, the medical patch 100 includes a release front layer 102 and an impermeable back layer 104. The release front layer 102 touches the skin of a user when the patch 100 is applied to the skin of the user. The release front layer 102 includes a central bandage area 106 that includes infused activated charcoal 1060 and is preferably slightly moistened with water before applying to the ailing body area of a user. A layer of recessed area 108 is disposed over the central bandage area 106, thereby allowing a user to apply a desired topical ointment, oil or any other medication to the recessed area 108. The activated charcoal 1060 can be mixed with the desired medication applied to the recessed area 108, thereby allowing a more effective treatment therapy for pain relief on the ailing area of the body. The activated charcoal 1060 of the central bandage area 106 aids and helps draw out impurities and infections from wounds and also helps in relieving pain.

In use, when the patch 100 is applied to joints for relief in arthritis pain, activated charcoal infused in the central bandage area 106 promotes circulation and draws out toxins from the joint that relieve arthritis pain, and soothes other symptoms such as stiffness and swelling. The ointment or other medication of the recessed area 108 also helps in relieving the user from pain and thus together with the activated charcoal, provides effective and quick pain relief on any part of body. The improved patch 100 enables a user to use a potent natural ingredient in the form of activated charcoal and an additional medication together for quick pain relief. The ointment applied on the recessed area 108 can be a charcoal-containing medicament which can be used in combination with the infused activated charcoal. Administration of the charcoal and other charcoal-containing medicament can be simultaneous, separate and/or sequential. The charcoal, in combination with another medication, can act additively or synergistically for relieving pain.

The impermeable backing layer 104 prohibits the medication and the activated charcoal from passing through the backing layer 104. It is to be appreciated that the backing layer 104 is visually exposed when the patch 100 is applied to the skin of the user. The backing layer 104 prevents the patch 100 from soiling clothes with medication. Further, the backing layer 104 can come in multiple colors, sizes and designs.

On both sides of the central bandage 106, the release front layer 102 includes adhesive coating areas 110, 112 that can be used for sticking the patch 100 on the body. The adhesive coating areas 110, 112 are optional in some of the embodiments of the present invention, and in such cases, the patch 100 can be attached to the skin using a bandage or medical tape. It should be appreciated that the improved patch 100 of the present invention enables the skin of the body to absorb the activated charcoal and the medication over a period of time. A user need not take a pill or an injection and the patch 100 provides a more comfortable option for taking some pain-relieving medications along with natural ingredients such as the activated charcoal. The transdermal administration by the improved patch 100 is useful for acute and chronic pains such as arthritis, and enables prolonged release of medications, which can improve patient adherence. The infused activated charcoal in the patch 100 can be based on the type of pain and the part of the body to which the patch 100 has to be applied.

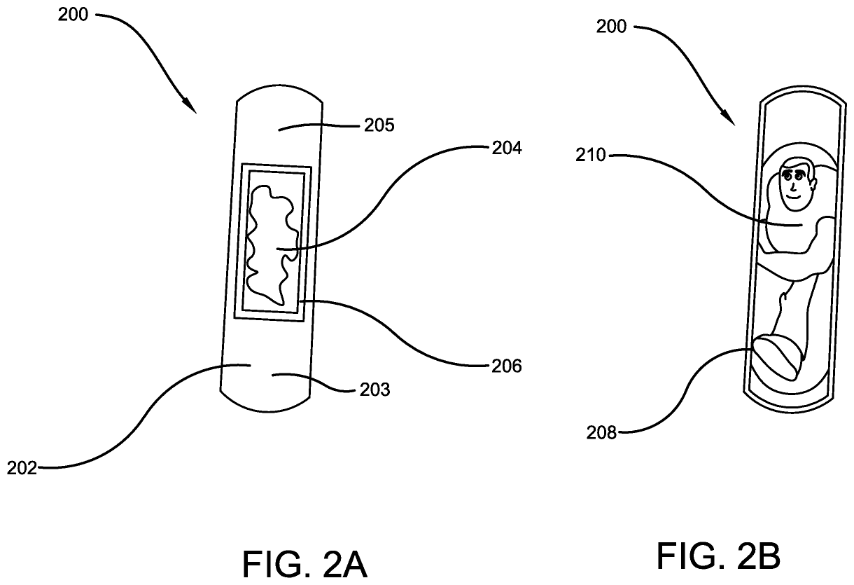

FIG. 2A illustrates a rear perspective view of another embodiment of the transdermal medical patch device of the present invention in accordance with the disclosed architecture. The present embodiment of the transdermal medical patch device can be in the form and shape of a bandage-type tape 200. The rear surface 202 of the bandage tape 200 includes adhesive layer areas 203, 205 that can be used for sticking the tape 200 to the skin of a user. The central bandage portion 204 is infused with activated charcoal and a recessed area 206 circumscribing the central bandage area 204 is configured to be infused with a medication such as buprenorphine, fentanyl or any other opioid pain medication. In the present embodiment, the opioid can be infused with from about 2 micrograms to about 10 micrograms per hour patches. Alternatively, the opioid medication can be infused with from about 6 micrograms to about 8 micrograms per hour patches. The bandage-type tape 200 can be used on any joint for effective relief from any pain, and especially from arthritis.

FIG. 2B illustrates a front perspective view of the another embodiment of the transdermal medical patch device of the present invention in accordance with the disclosed architecture. The bandage tape 200 includes an impermeable top layer 208 that is waterproof, and prohibits water from reaching the opioid medication or the charcoal. The top layer 208 also acts as a barrier for the medication from foreign substances, and can include any design or appearance 210 as per the preference of the user without compromising the medical efficacy of the medical strap tap 200.

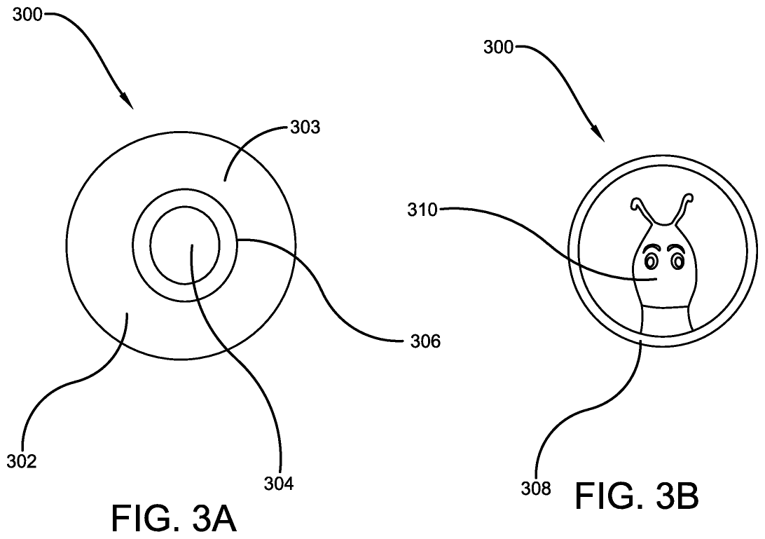

FIG. 3A illustrates a rear perspective view of yet another embodiment of the transdermal medical patch device of the present invention in accordance with the disclosed architecture. The present embodiment of the transdermal medical patch device is in the form of a circular coin-size tape 300. The rear surface 302 of the circular tape 300 includes an adhesive layer 303 that is used for sticking the tape 300 to the skin of a user. The central bandage portion 304 can be infused with activated charcoal and a recessed area 306 circumscribes the central bandage area 304 and can be infused with an opioid medication. Any pain medication, as per the preference of the user, can be applied to the recessed area 306 by the user himself. The recessed area 306 is configured to absorb the medication which is then topically delivered to the body of the user. The tape 300 can be used on any joint for effective relief from any pain, and especially from arthritis.

FIG. 3B illustrates a front perspective view of the yet another embodiment of the transdermal medical patch device of the present invention in accordance with the disclosed architecture. The circular tape 300 includes an impermeable top layer 308 that is waterproof and prohibits water from reaching the opioid medication or the charcoal. The top layer 308 can also act as a barrier for the medication from foreign substances, and can include any design or appearance 310 as per the preference of the user without compromising the medical efficacy of the medical strap tape 300.

The improved medical patch of various embodiments of the present invention does not include smelly chemicals, irritating ingredients for the skin or leave rashes on the user's skin. In some embodiments, juice or extract of proven pain-relieving ingredients like turmeric, ginger, magnesium and chamomile, among others, can be applied on the recessed area for topically applying to the body.

The medical patch is available in a variety of shapes and sizes, and can be configured in coin shape, bandage shape, card shape, wide strips or any other geometrical shape with infused medication in a recessed area. Alternatively, the medical patch can be applied without infused medications.

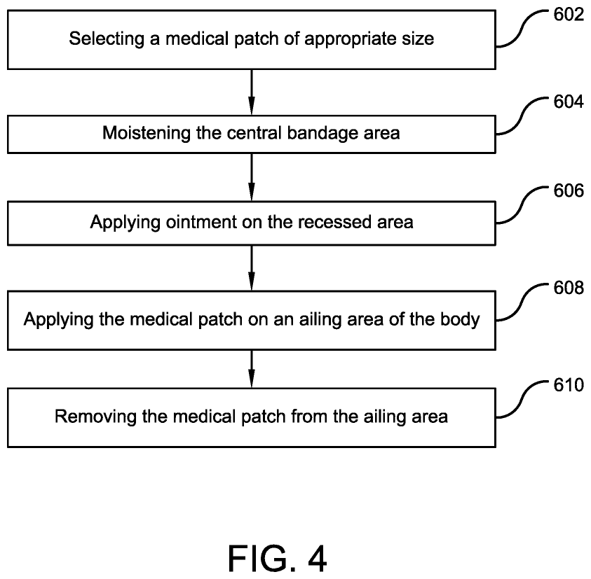

FIG. 4 illustrates a flow diagram showing exemplary steps performed for a method of providing relief from pain using the medical patch of the present invention in accordance with the disclosed architecture. It should be understood that the present embodiment states the basic flow performed by a user and additional actions may be used to add additional processes to the basic flow. Initially, a medical patch of the appropriate size and configuration is selected by a user for applying (Block 602). Then, the central bandage area of the medical patch device is moistened to activate the dried charcoal (Block 604). Thereafter, based on type and intensity of the pain, an ointment is applied on the recessed area of the medical patch (Block 606). Then, for providing relief from the pain, the medial patch is applied to an ailing area of the body in a non-invasive manner (Block 608). Finally, after a desired duration of application of the patch on the ailing area, the patch is removed, and selectively can be replaced with a new patch or can be reapplied after infusing medication and charcoal (Block 610).

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “improved medical patch”, “medical patch”, “bandage tape”, “circular coin size tape”, “patch”, “improved patch”, and “pain-relieving patch” are interchangeable and refer to the improved medical patch 100, 200, 300 of the present invention.

Notwithstanding the forgoing, the improved medical patch 100, 200, 300 of the present invention can be of any suitable size and configuration as is known in the art without affecting the overall concept of the invention, provided that it accomplishes the above-stated objectives. One of ordinary skill in the art will appreciate that the size, configuration and material of the improved medical patch 100, 200, 300 as shown in the FIGS. are for illustrative purposes only, and that many other sizes and shapes of the improved medical patch 100, 200, 300 are well within the scope of the present disclosure. Although the dimensions of the improved medical patch 100, 200, 300 are important design parameters for user convenience, the improved medical patch 100, 200, 300 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A medical patch applied to a skin of a user for relieving a chronic ache or pain, the medical patch comprising:
a release front layer and an impermeable backing layer;
wherein said release front layer touches the skin of the user when said medical patch is applied to the skin of the user;
wherein said release front layer comprises a central bandage area infused with an activated charcoal to draw out impurities and infections from a wound on the skin; and
further wherein said central bandage area is circumscribed by a recessed area for holding a topical ointment.

2. The medical patch of claim 1, wherein said impermeable backing layer prohibits said activated charcoal and said topical ointment from passing therethrough.

3. The medical patch of claim 2, wherein said backing layer is visually exposed when said medical patch is applied to the skin of the user.

4. The medical patch of claim 1, wherein said release front area comprises a first adhesive area and a second adhesive area.

5. The medical patch of claim 4, wherein said first adhesive area extends from a first edge of said central bandage area to a first terminal edge of said medical patch and said second adhesive area extends from a second edge of said central bandage area to a second terminal edge of said medical patch.

6. The medical patch of claim 5, wherein said first adhesive area and said second adhesive area adhere to the skin of the user.

7. The medical patch of claim 6, wherein said activated charcoal is moistened with a water before applying to the skin of the user.

8. The medical patch of claim 7, wherein said topical ointment is an extract selected from a group consisting of a pain-relieving ingredient, a turmeric, a ginger, a magnesium and a chamomile.

9. The medical patch of claim 7, wherein said topical ointment is an opioid infusing from about 2 micrograms to about 10 micrograms per hour to the skin of the user.

10. A transdermal medical patch applied to a skin of a user for relieving a chronic ache or pain, the transdermal medical patch comprising:
a release front layer that touches the skin of a user when said transdermal medical patch is applied to the skin of the user, wherein said release front layer comprises a central bandage area infused with an activated charcoal to draw out impurities and infections from the skin of the user, and further wherein said central bandage area circumscribed by a recessed area for holding a topical ointment; and
an impermeable backing layer, wherein said recessed area is circumscribed by an adhesive area extending from said recessed area to a terminal edge of said transdermal medical patch, and further wherein said adhesive area adheres to the skin of the user.

11. The transdermal medical patch of claim 10, wherein said impermeable backing layer prohibits said activated charcoal and said topical ointment from passing therethrough.

12. The transdermal medical patch of claim 11, wherein said impermeable backing layer is visually exposed when said transdermal medical patch is applied to the skin of the user.

13. The transdermal medical patch of claim 12, wherein said activated charcoal is moistened with a water before applying to the skin of the user.

14. The transdermal medical patch of claim 13, wherein said topical ointment is an extract selected from a group consisting of a pain-relieving ingredient, a turmeric, a ginger, a magnesium and a chamomile.

15. The transdermal medical patch of claim 13, wherein said topical ointment is an opioid infusing from about 2 micrograms to about 10 micrograms per hour to the skin of the user.

16. A method for relieving an ache or a pain using a transdermal medical patch applied to a skin of a user, the method comprising the steps of:
forming the transdermal medical patch including a release front layer and an impermeable back layer;
wherein said release front layer includes a central bandage area infused with an activated charcoal to draw out impurities and infections from the skin;
wherein said central bandage area circumscribed by a recessed area for holding a topical ointment;
wherein said recessed area circumscribed by an adhesive area;
wherein said adhesive area extending from said recessed area to a terminal edge of said medical patch; and
applying said transdermal medical patch to the skin of the user, wherein said adhesive layer and said release front layer adhered to the skin of a user.

17. The method of claim 16, wherein said impermeable backing layer prohibits said activated charcoal and said topical ointment from passing therethrough.

18. The method of claim 16 further comprising a step of moistening said activated charcoal with a water before applying to the skin of the user.

19. The medical patch of claim 16, wherein said topical ointment is an extract selected from a group consisting of a pain-relieving ingredient, a turmeric, a ginger, a magnesium and a chamomile.

20. The medical patch of claim 16, wherein said topical ointment is an opioid infusing from about 2 micrograms to about 10 micrograms per hour to the skin of the user.
